(19) United States
(12) Patent Application Publication
(10) Pub. No.: US 2004/0197302 A1
(43) Pub. Date: Oct. 7, 2004

(54) Prepolymeric materials for site specific delivery to the body

(76) Inventors: Christopher H. Porter, Woodenville, WA (US); Robert Ziebol, Blaine, MN (US)

Correspondence Address: Gerald F. Swiss, Foley & Lardner LLP, Three Palo Alto Square, 3000 El Camino Real, Suite 100, Palo Alto, CA, 94306-2121, US

(21) Appl. No.: 10/687,545

(22) Filed: Oct. 15, 2003

Related U.S. Application Data
(60) Provisional application No. 60/418,251, filed on Oct. 15, 2002.

(51) Int. Cl. A61K 31/74

(52) U.S. Cl. 424/78.31

(57) ABSTRACT

Disclosed are compositions for site specific delivery in the body including diseased vasculature (e.g., aneurysmal sacs, arteriovenous malformations, etc.), body lumens such as the vas deferens and fallopian tubes, cavities created in vivo for the purpose of tissue bulking, and the like. Also disclosed are methods employing such compositions as well as kits comprising such compositions.

### CROSS-REFERENCE TO RELATED APPLICATIONS

This application claims the benefit under 35 U.S.C. § 119(e) of U.S. Provisional Application 60/418,251, filed Oct. 15, 2002, which is hereby incorporated by reference in its entirety.

### BACKGROUND OF THE INVENTION

1. Field of the Invention

This invention relates to compositions for site specific delivery in the body including diseased vasculature (e.g., aneurysmal sacs, arteriovenous malformations, etc.), body lumens such as the vas deferens and fallopian tubes, cavities created in vivo for the purpose of tissue bulking and the like. This invention also relates to methods employing such compositions as well as kits comprising such compositions.

The compositions of this invention comprise a prepolymeric material which thickens and/or solidifies in situ in the presence of an exogenous trigger and a sufficient amount of a rheological modifier to permit the composition to exhibit thixotropic behavior. This thixotropic behavior permits the compositions to exhibit high viscosities under static conditions while the prepolymeric material is solidifying or thickening in vivo.

### REFERENCES

The following publications and patents are cited in this application as superscript numbers:

1. Porter, Methods and Apparatus for Delivering Materials to the Body, International Patent Application Publication No. WO 02/087416 published 7 Nov. 2002

2. Evans, et al., Embolizing Compositions, U.S. Pat. No. 5,695,480, issued Dec. 9, 1997

3. Askill, et al., U.S. Pat. No. 5,855,208, Methods for Draping Surgical Incision Sites Using a Biocompatible Prepolymer, issued Jan. 5, 1999.

4. Okada, et al., Intravascular Embolizing Agent Containing Angiogenesis-Inhibiting Substance, U.S. Pat. No. 5,202,352, issued on Apr. 13, 1993.

5. Wallace, et al., Methods for Treating Urinary Incontinence in Mammals, U.S. Pat. No. 6,569,417, issued May 27, 2003.

6. Greff, et al., Methods for Soft Tissue Augmentation in Mammals, U.S. Pat. No. 6,231,613, issued May 15, 2001.

7. Wallace, et al., Methods for Treating Urinary Reflux, U.S. Pat. No. 5,958,444, issued Sep. 28, 1999.

8. Silverman, et al., Method for Treating Gastroesophageal Reflux Disease and Apparatus for Use Therewith, issued May 29, 2001.

All of the above publications and patents are herein incorporated by reference in their entirety to the same extent as if each individual publication or patent was specifically and individually indicated to be incorporated by reference in its entirety.

2. State of the Art

Compositions for delivery into the body including body cavities are well known in the art. Such compositions have included reactive substances optionally in the presence of a liquid (e.g., solvent) and a contrast agent. Reactive substances include both reactive prepolymers which polymerize in vivo in the absence of an external trigger as well as those which polymerize in the presence of a trigger. ^{1,2}

The optional biocompatible solvent can be employed to render the composition more lubricous during delivery and/or to dissolve the prepolymer (if the prepolymer is not liquid) and/or the contrast agent. In either case, when the prepolymer is delivered in vivo it reacts thereby thickening or solidifying to provide for a solid mass which can act as, e.g., a drug delivery depot, an embolic mass, etc.

One group of such compositions recently receiving extensive evaluations are embolic compositions that, again, are well known in the art. Representative prepolymeric embolic compositions include those found in Porter¹ and Evans, et al.² Of these compositions, those showing most promise as embolic agents comprise a prepolymeric material, an optional solvent and a contrast agent. Such compositions are typically employed for a variety of purposes including the treatment of tumors, the treatment of vascular lesions such as aneurysms, arteriovenous malformations (AVM), arteriovenous fistula (AVF), uncontrolled bleeding and the like.

Embolization of blood vessels is preferably accomplished via catheter techniques that permit the selective placement of the catheter at the vascular site to be embolized. In this regard, recent advancements in catheter technology as well as in angiography now permit neuroendovascular intervention including the treatment of otherwise inoperable lesions. Specifically, development of microcatheters and guide wires capable of providing access to vessels as small as 1 mm in diameter allows for the endovascular treatment of many lesions.

When using embolizing compositions for filling cavities of the body, especially brain aneurysms, it is highly desirable that the filling material, after delivery, not flow out of the cavity. It can be stated that the higher the viscosity of the fluid in the aneurysm, the better or more effective the treatment since complications arising from out flow are mitigated. However, prepolymers are typified by very low viscosities. Moreover, rapid polymerization of the prepolymer in vivo can lead to high heats of reaction which can damage tissue as well as entrap the catheter tip in the formed mass.

For example, running or flow of the composition from its intended delivery site is of concern as well as the fact that when water insoluble contrast agents are employed, retention of these agents in suspension during delivery from the catheter requires shaking of the composition prior to use coupled with the use of particles of sufficiently small size to mitigate against settling.²

As to the use of prior art compositions for filling other body cavities, similar problems arise. That is to say that the composition should have a sufficient high viscosity to exhibit site selective placement in the body while at the same time being sufficient fluid as to permit the clinician to readily deliver the material in vivo. Low viscosity materials can continue to flow when placed in vivo and can result in delivery of the composition to unintended sites.

As such, there is an ongoing need to provide a prepolymeric composition that has a very high viscosity when placed in vivo such that subsequent solidification is site specific in the body.

### SUMMARY OF THE INVENTION

This invention is directed to novel compositions for site specific delivery into the body such as filling cavities in the body, particularly aneurysms, and methods of treatment related thereto. The compositions of this invention have the particular advantage of exhibiting a high static viscosity such that they exhibit site selective placement in vivo and a low viscosity during delivery to permit injection of these compositions under acceptable delivery pressures.

In one embodiment, this application is directed to a composition comprising a prepolymeric material which thickens and/or solidifies in situ in the presence of a exogenous trigger and a sufficient amount of a rheological modifier to permit the composition to exhibit thixotropic behavior prior to completion of the thickening and/or solidifying the prepolymeric material.

In a further embodiment, the composition further comprises a contrast agent. The contrast agent can be either water soluble or water insoluble contrast agents with preferred agents being water insoluble. Examples of water insoluble contrast agents include tantalum, tantalum oxide, tungsten, gold, platinum and barium containing compounds such as barium sulfate. Examples of water soluble contrast agents include metrizamide, iopamidol, jothalamate sodium, jodomide sodium, and meglumine.

The compositions of this invention can also comprise other optional components such as plasticizers, surfactants, and the like. Examples of plasticizers include aromatic esters, alkyl esters, phthalate esters, citrate esters, glycerol esters, plant derived oils, animal derived oils, silicone oils, iodinated oils, vitamins A, C, E and acetates and esters thereof, and mixtures thereof.

This invention is also directed to a method for delivering composition of this invention to mammalian patients. These methods comprise inserting an appropriate delivery device at a targeted site in the patient and then administering via the delivery device a composition of this invention as described above under such conditions that a mass is formed in vivo.

The delivery methods described herein can be employed to embolize blood vessels, to bulk tissue, to provide a depot for drug delivery, and the like.

For example, the compositions described herein can further comprise a radioactive material such that the composition can be used to ablate diseased tissue such as tumors, arteriovenous malformations, and the like. Suitable radioactive materials include, for example, of ⁹⁰yttrium, ¹⁹²iridium, ¹⁹⁸gold, ¹²⁵iodine, ¹³⁷cesium, ⁶⁰cobalt, ⁵⁵cobalt, ⁵⁶cobalt, ⁵⁷cobalt, ⁵⁷magnesium, ⁵⁵iron, ³²phosphorous, ⁹⁰strontium, ⁸¹rubidium, ²⁰⁶bismuth, ⁶⁷gallium, ⁷⁷bromine, ¹²⁹cesium, ⁷³selenium, ⁷²selenium, ⁷²arsenic, ¹⁰³palladium, ²⁰³lead, ¹¹¹indium, ⁵²iron, ¹⁶⁷thulium, ⁵⁷nickel, ⁶²zinc, ⁶²copper, ²⁰¹thallium and ¹²³iodine.

The compositions can also further comprise a medicament such as an angiogenesis inhibiting compound, a steroidal or non-steroidal anti-inflammatory agent, a thrombotic agent, and the like. The invention also contemplates a method for delivering said compositions comprising the medicament.

Methods for embolizing a blood vessel are preferably accomplished by delivering via a catheter into a vascular site to be embolized a composition of this invention. Such methods preferably comprise inserting the distal end of the catheter in the selected vascular site, delivering via the catheter a composition of this invention under conditions wherein a mass is formed which embolizes the blood vessel.

Methods for bulking tissue are preferably accomplished by delivering via a delivery device at the tissue site to be bulked a composition of this invention. Such methods preferably comprise inserting the delivery device into the selected tissue, delivering via the device a composition of this invention under conditions wherein a solid mass is formed which bulks the tissue.

Suitable tissue sites for bulking include the suburethral tissue, the periurethreal tissue, soft tissue and sphincters such as the esophageal sphincter.

Suitable delivery devices includes needles, syringes, catheters, and the like.

This invention is also directed to a kit of parts comprising a) a composition comprising a prepolymeric material which thickens and/or solidifies in situ in the presence of a exogenous trigger, a sufficient amount of a rheological modifier to permit the composition to exhibit thixotropic behavior, and optionally contrast agent; and b) a delivery device.

The compositions and methods of this invention provide one or more of the following advantages relative to non-rheologically modified compositions:

i) when a contrast agent is employed, the compositions require little if any shaking prior to use since the rheological modifier acts as a suspending agent;

ii) the high viscosity of the rheologically modified composition under static conditions permits site specific delivery in vivo including improved start-stop characteristics during delivery (the composition will not tend to flow after the pressure has been removed thereby reducing drool) and more uniform and predictable set-up in vivo. In this regard, the rheological modifier acts as a matrix for defining the site of polymerization and/or solidification of the prepolymer thereby minimizing flow from the intended site of delivery in vivo; and

iii) during shear stress the rheologically modified composition acts as a piston at the interface of this composition and the previously delivered composition, particularly through a catheter or other delivery device, and effectively removes the prior delivered composition from the delivery device with minimal mixing of the two compositions.

Additional advantages and novel features of the invention will be set forth in part in the description which follows, and in part will become apparent to those skilled in the art upon examination of the following, or may be learned by practice of the invention.

### BRIEF DESCRIPTION OF THE DRAWINGS

FIG. 1 illustrates the non-Newtonian behavior of a composition of this invention wherein a sufficient amount of fumed silica is contained in the composition to permit it to exhibit thixotropic behavior.

### DETAILED DESCRIPTION OF THE INVENTION

As discussed above, this invention is directed to novel compositions for filling cavities in the body, particularly aneurysms, and methods of treatment related thereto.

Before this invention is described in detail, it is to be understood that unless otherwise indicated this invention is not limited to any particular composition, as such may vary. It is also to be understood that the terminology used herein is for the purpose of describing particular embodiments only and is not intended to limit the scope of the present invention. It must be noted that as used herein and in the claims, the singular forms “a,” “an” and “the” include plural referents unless the context clearly dictates otherwise. In this specification and in the claims which follow, reference will be made to a number of terms which shall be defined to have the following meanings:

The term “biocompatible” means that the material or substance described is non-toxic at the concentrations employed and is substantially non-immunogenic again at the concentrations employed.

The term “a prepolymeric material which thickens and/or solidifies in situ in the presence of a exogenous trigger” refers to any biocompatible material which form a mass in vivo by reactive mechanisms employing at least one component which is exogenously provided. Such prepolymeric materials include, by way of example only, acrylates, methacrylates, acrylamides, methacrylamides, styrenes, vinyl acetate, acrylonitrile, mixtures thereof with one another as well as mixtures with maleic acid, urethane, urethane carbonates, silicone, epoxy, and the like. Examples include urethane acrylates and epoxy acrylates from Sartomer, Exton, Pa., USA, urethane acrylates from Polymer Systems Corp., Washington (i.e., Purelast®), acrylate and methacrylate epoxies and urethanes from Echo Resins and Laboratories, Inc. (Versailles, Mo.); epoxy and urethane acrylates available from Cargill, Inc. (Minneapolis, Minn.), radiation curable acrylic resins from P.D. George Co., St. Louis, Mo., USA (i.e., Tritherm®, Terasod®, Pedigree®, and Soderite®), urethane olefin precursors from Hampshire Chemical Company, Lexington, Mass., USA (i.e., Hypol® 2000), Monomer-Polymer and Dajac Laboratories, Inc., Feasterville, Pa., USA (i.e., Photomer® 6230), Henkel Corporation, Germany, (i.e., Photomer® 6264), and silicone acrylate from NuSil (Carpenteria, Calif.).

Other examples of prepolymers suitable for use in this invention are set forth in Askill, et al.³

In any event, the prepolymer is adapted to at least partially polymerize in situ in the presence of an exogenous trigger when the trigger is introduced to the body site where the prepolymer has been placed. Prepolymers not contemplated by this invention are cyanoacrylates which polymerize in the presence of endogenous proteins.

The term “exogenous trigger” refers to triggers exogenously introduced into the body site where the prepolymer has been place and which, when activated, initiates or furthers formation of a mass from the prepolymer. Examples of suitable triggers include, for instance, light (such as UV, IR and visible light), electromagnetic radiation, ultrasound, mechanical force, magnetic fields, heating or cooling, the introduction of a salt or catalyst, an acid or a base, another reactive component and the like. Triggering of the reactive mechanisms to effect mass formation are well known in the art.

The term “biocompatible contrast agent” or “contrast agent” refers to a biocompatible radiopaque material capable of being monitored during injection into a mammalian subject by, for example, radiography. In the methods of this invention, the contrast agent is preferably water insoluble (i.e., has a water solubility of less than 0.01 mg/ml at 20° C.). Examples of biocompatible water-insoluble contrast agents include tantalum, tantalum oxide, and barium containing compounds, such as barium sulfate, each of which is commercially available in the proper form for in vivo use. Other biocompatible water-insoluble contrast agents include gold, tungsten, and platinum. Preferred biocompatible water-insoluble contrast agents are those having an average particle size of about 10 μm or less. Water soluble contrast agents are also suitable for use herein and include, for example, metrizamide, lipidol and the like. Preferably, the biocompatible contrast agent employed does not cause a substantial adverse inflammatory reaction when employed in vivo.

The term “thixotropic properties” or “thixotropic behavior” refers to the shear thinning capacity of a composition which correlates with a non-Newtonian viscosity relationship such that the composition flows more easily under higher shear rates. Stated another way, the apparent viscosity of the composition decreases with increased shear rate. Another exemplified behavior would be that of a Bingham plastic. A Bingham plastic is a material that has infinite viscosity when no shear rate is applied but flows once shear rate is applied. Compositions under shear or dynamic conditions should exhibit an apparent viscosity of less 10,000 centipoise (cP) at 40° C. and the viscosity under static conditions should be at least 1.5 times over the dynamic viscosity.

The term “biocompatible liquid” refers to a material liquid at least at body temperature of the mammal.

When the biocompatible liquid is employed to dissolve the soluble rheological modifier (as defined below), the biocompatible liquid is employed as a solvent and is sometimes described herein as a “biocompatible solvent”. Suitable biocompatible solvents include, by way of example, ethyl lactate, dimethylsulfoxide (DMSO), analogues/homologues of dimethylsulfoxide, ethanol, acetone, and the like. Aqueous mixtures with the biocompatible solvent can also be employed, provided that the amount of water employed is sufficiently small that the dissolved polymer mass upon contact with blood or other bodily fluid. Preferably, the biocompatible solvent is dimethylsulfoxide.

When the biocompatible liquid is employed as a lubricous agent, the solubility of the rheological modifier is not essential and suitable solvents such as water, oils, emulsions, and the like can be used.

The term “embolizing” refers to a process wherein a material is injected into a blood vessel which, in the case of, for example, aneurysms, fills or plugs the aneurysmal sac and/or encourages clot formation so that blood flow into the aneurysm ceases. In the case of AVMs, a plug or clot is formed to control/reroute blood flow to permit proper tissue perfusion. In the case of a vascular site, the vascular site is filled to prevent blood flow there through. Embolization of the blood vessel is important in preventing and/or controlling bleeding due to lesions (e.g., organ bleeding, gastrointestinal bleeding, vascular bleeding, and bleeding associated with an aneurysm). In addition, embolization can be used to ablate diseased tissue (e.g., tumors, etc.) by cutting off the diseased tissue's blood supply.

The term “encapsulation” as used relative to the contrast agent being encapsulated in the polymer mass, does not infer any physical entrapment of the contrast agent within the mass, much as a capsule encapsulates a medicament. Rather, this term is used to mean that an integral, coherent mass forms which does not separate into individual components.

The term “rheology” refers to the science of flow and deformation of matter, and describes the interrelation between force, deformation, and time.

The term “rheological modifier” as used herein, refers to a component which, when added to a composition, imparts high viscosity to the composition under static conditions, yet permits the composition to flow freely under shear stress. Compositions of this invention may use one or more rheological modifiers, including combinations of rheological modifiers. As used herein, rheological modifiers are generally classified as a non-particulate rheological modifier or a particulate rheological modifier. The preferred rheological modifier is fumed silica.

The term “non-particulate rheological modifier” as used herein, refers to a rheological modifier which can be solubilized or suspended in the biocompatible liquid employed. Soluble rheological modifiers include, but are not limited to, polyacrylates, polyalkenes, polyalkyl oxides, polyamides, polycarbonates, cellulosic polymers and copolymers thereof, polydienes, polyesters, polymethacrylates, polysiloxanes, polystyrenes, polyurethanes, polyvinyl ethers, polyvinyl esters, Carbopol, acrylic polymers, cross-linked acrylic polymers, hydroxypropylcellulose, hydroxypropylmethylcellulose, oxidized polyethylene and their copolymers, polyethylene oxide, polyvinylpyrrolidone, associative thickeners, Carrageenan, carboxymethylcellulose, sodium hydroxyethylcellulose, hydroxyethylcellulose, methylcellulose, Guar, Guar derivatives, Locust Bean Gum, Xanthan Gum, and mixtures thereof.

The term “particulate rheological modifier” as used here, refers to a rheological modifier which is mineral-based. Particulate rheological modifiers include, but are not limited to, silacatious earths, bentonite, organoclays, water-swellable clays, such as lapenite, and silicas such as fumed silica and precipitated, calcium carbonate, titanium dioxide, laminate, titanium oxide, zinc oxide, hydroxyappetite, carbon beads, dispersed fiber, magnetic materials and mixtures thereof. Preferably, the particulate rheological modifier is fumed silica.

The term “shear stress” refers to the ratio of force to area across, for example, a liquid. The liquid's response to the applied shear stress is to flow. A velocity gradient forms that gives the “shear rate.” The viscosity of the liquid is the ratio of shear stress to shear rate. Newtonian fluids exhibit a linear relationship between shear stress and shear rate, making viscosity independent of the applied shear conditions. Non-Newtonian fluids do not exhibit the linear relationship between shear stress and shear rate. An example would be a Bingham plastic. “Shear-Thinning” or “Pseudoplasticity” is a common non-Newtonian flow, where viscosity decreases as shear increases. In a less common non-Newtonian flow, “Shear-Thickening” or “Dilatancy,” viscosity increases as shear increases. The biocompatible compositions of the instant invention exhibit Pseudoplastic flow.

“Static conditions” as used herein means that the shear rate applied is at most about 1 s⁻¹.

“Surfactants” are those substances which enhance flow and/or aid dispersion by reducing surface tension when dissolved in water or water solutions, or that reduce interfacial tension between two liquids, or between a liquid and a solid. Surfactants also impede the interaction between the rheological modifier and other components of the system. This allows a more fully developed rheological modified system. Surfactants may be anionic, cationic, and nonionic. Surfactants include detergents, wetting agents, and emulsifiers. Suitable cationic surfactants include organic amines and organic ammonium chlorides (e.g., N-tallow trimethylene diamine diolealate and N-alkyl trimethyl ammonium chloride) and the like. Suitable anionic surfactants include, by way of example sulfosuccinates, carboxylic acids, alkyl sulfonates, octoates, oleates, stearates, and the like. Suitable nonionic surfactants, include by way of example, bridging molecules discussed above, Tritons, Tweens, Spans and the like.

The term “viscosity” refers to a substance's ratio of shearing stress to rate of shear.

Compositions

The biocompatible rheologically-modified compositions described herein are prepared by conventional methods. For illustrative purposes only, compositions comprising a liquid biocompatible prepolymer, a rheological modifier, and a water insoluble contrast agent are described. It is understood that the omission of the contrast agent from the compositions described herein would entail merely eliminating that aspect during preparation. In any event, these compositions can be prepared by in a first step combining sufficient amounts of a biocompatible prepolymer and a contrast agent at ambient conditions or at moderately elevated temperatures while mixing to achieve a uniform suspension.

After addition of the polymer and contrast agent, the rheological modifier is added under ambient conditions, preferably under inert atmosphere, for example, an argon atmosphere. If a particulate rheological modifier is used, the composition is initially stirred at low RPM (less than about 1000 RPM) to wet the surface of the rheological modifier. Once wetted, the stir rate is increased to a peripheral tip speed of from about 5 m/sec to about 26.5 m/sec. The tip speed should be maintained until no granular material is evidenced in the composition. When soluble rheological modifiers are used, the composition need not be stirred at low RPM and these modifiers are easily added to the composition.

The viscosity of the composition is modified by the addition of one or more rheological modifiers or a mixture thereof. The addition of the rheological modifier(s) provides a composition exhibiting a relative decrease in the viscosity under shear stress as compared to its viscosity under static conditions.

A particularly preferred rheologically-modified composition comprises a solution of about 3 to about 12 weight percent of biocompatible prepolymer, about 20 to about 55 weight percent of a contrast agent, more preferably about 37 to about 40 percent contrast agent and about 3 to about 12 percent rheological modifier. All of the above percentage values are based on the total weight of composition. Optionally, a biocompatible liquid can be added to enhance one or more of the properties of the composition, e.g., lubricity.

Other Components

Surfactants can be optionally employed in the biocompatible rheologically-modified composition. When employed, surfactants maintain dispersion of the rheological modifier and the contrast agent in the liquid. Surfactants also impede the interaction between the rheological modifier and other components of the system. This allows for more fully developed rheologically-modified systems.

When surfactants are employed, a preferred biocompatible rheologically-modified composition comprises about 3 to about 12 weight percent of biocompatible polymer, about 20 to about 55 weight percent of a contrast agent, preferably, about 37 to about 40 percent of contrast agent about 3 to about 12 percent rheological modifier, and about 0.1 to about 1.0 weight percent of the rheological modifier is surfactant. Again, all of the above percentage values are based on the total weight of composition.

Methods

The compositions described above can then be employed in methods for site specific delivery into the body including filling of body cavities. For example, the compositions described above can then be employed in methods for the catheter assisted intra-vascular embolization of mammalian blood vessels. The methods of this invention are employed at intra-vascular sites wherein preferably blood flow during the embolization process at the vascular site to be treated is attenuated, but not arrested. Attenuation of blood flow arises by placement of the catheter into the vascular site, wherein blood flow therethrough is reduced. For example, a microballoon may be employed to attenuate blood flow. In the methods of this invention, a sufficient amount of the biocompatible rheologically-modified composition is introduced into the vascular site via, for example, a catheter under fluoroscopy so that upon mass formation, the vascular site is embolized. The particular amount of composition employed is dictated by the total volume of the vasculature to be embolized, the concentration of prepolymer in the composition, the rate of mass formation, etc. Such factors are well within the skill of the art.

In the catheter delivery methods described herein, a small diameter medical catheter (i.e., microcatheter) having a diameter typically from about 1 mm to about 3 mm is employed. The particular catheter employed is not critical, provided that catheter components are compatible with the composition (i.e., the catheter components will not readily degrade in the composition). In this regard, it is preferred to use polyethylene in the catheter components because of its inertness in the presence of the composition described herein. Other materials compatible with the compositions can be readily determined by the skilled artisan and include, for example, other polyolefins, fluoropolymers (e.g., polytetrafluoroethylene, perfluoroalkoxy resin, fluorinated ethylene propylene polymers, etc.), silicone, etc. The specific polymer employed is selected relative to stability in the presence of the solvent and preferably has lubricious properties.

Alternatively, the compositions of this invention can be used for tissue bulking or augmentation. For example, injection of the material into the periurethral tissue to form a solid mass can be used to treat incontinence in a manner similar to that described by Wallace, et al.⁵ Further, the compositions of this invention can be used to augment soft tissue in a manner similar to that described by Greff, et al.⁶ The compositions of this invention can also be used to augment the suburethral tissue in mammals in order to treat urinary reflux as described by Wallace, et al.⁷ Augmentation of sphincters can be achieved in a manner similar to that described by Silverman, et al.⁸

Still further, the compositions of this invention can be used for the site specific delivery of a medicament or other material, e.g., a radioactive material, to a selected location in the body. Such medicaments can include anti-angeogenesis materials as described, for example, by Okada, et al.⁴ Other medicaments can include steroidal and non-steroidal anti-inflammatory agents, thrombotic agents and the like. Radioactive materials can be site specific delivered for the ablation of diseased tissue such as tumors, arteriovenous malformations, and the like.

Utility

The compositions and methods described herein are useful for site specific delivery of a composition into a mammalian body. The composition can be used for instance in the embolization of mammalian blood vessels which, in turn, can be used to prevent/control bleeding (e.g., organ bleeding, gastrointestinal bleeding, vascular bleeding, bleeding associated with an aneurysm) or to ablate diseased tissue (e.g., tumors, etc.). Accordingly, the invention finds use in human and other mammalian subjects requiring embolization of blood vessels.

The compositions have further utility in bulking soft tissue, sphincters lacking sufficient muscular tone to operate effectively, uretheral and periuretheral tissue and the like.

It is contemplated that the compositions can be employed as a carrier for a compatible, pharmaceutically-active compound wherein this compound is delivered in vivo for subsequent release. Such compounds include by way of example only antibiotics, anti-inflammatory agents, chemotherapeutic agents, anti-angiogenic agent, radioactive agents, growth factors and the like.

The following examples are set forth to illustrate the claimed invention and are not to be construed as a limitation thereof.

### EXAMPLES

Unless otherwise stated all temperatures are in degrees Celsius. Also, in these examples and elsewhere, abbreviations have the following meanings:

DMSO=dimethylsulfoxide

EH5=fumed silica having a surface area of approximately 380 m²/g (BET)

g=gram

cP=centipoise

RPM=revolution per minute

mm=millimeter

kg=kilogram

Equipment

Unless otherwise indicated, the following equipment was employed in the examples below:

1. Waring Blender (17,900 RPM and 21,300 no-load speed)

2. Viscometer—Brookfield, RVDV II+ (Brookfield Engineering, Middleboro, Mass.)

3. T-bar spindle—Brookfield (Brookfield Engineering, Middleboro, Mass.)

4. Helipath stand—Brookfield (Brookfield Engineering, Middleboro, Mass.)

5. Cowles disperser with a 2 inch blade with variable speed mixer (Morehouse-Cowles, Fullerton, Calif.)

The capillary rheometer used in this invention was constructed in the laboratory; however, a suitable rheometer may be purchased from Qualitest (Ft. Lauderdale, Fla.).

Compositions

The silica used in the examples presented below was obtained from Cabot Corporation. The tantalum is Q2 Grade NRC Capacitor grade tantalum metal powder from HC Starck (Newton, Mass.). The DMSO is USP grade.

### Example 1

The purpose of this example is to demonstrate the preparation of a composition of this invention that is suitable, in one embodiment, for embolizing an aneurysm.

In a beaker, a suitable amount 2-hydroxy methacrylate (available from Polysciences, Warrington, Pa.) was added. In a blender on low, containing the prepolymer. Fumed silica (6.7 weight percent of the total composition of EH5) was added to the vortex over approximately 2.5 minutes. After the addition of the last of the silica, the blender was run for an additional 15 seconds.

The viscosity of this composition of this invention was tested by pre-warming the viscometer to 37° C. and adding the above composition in the viscometer. In order to allow for equilibrium of the viscometer, the composition sat in the non-running viscometer for 15 minutes.

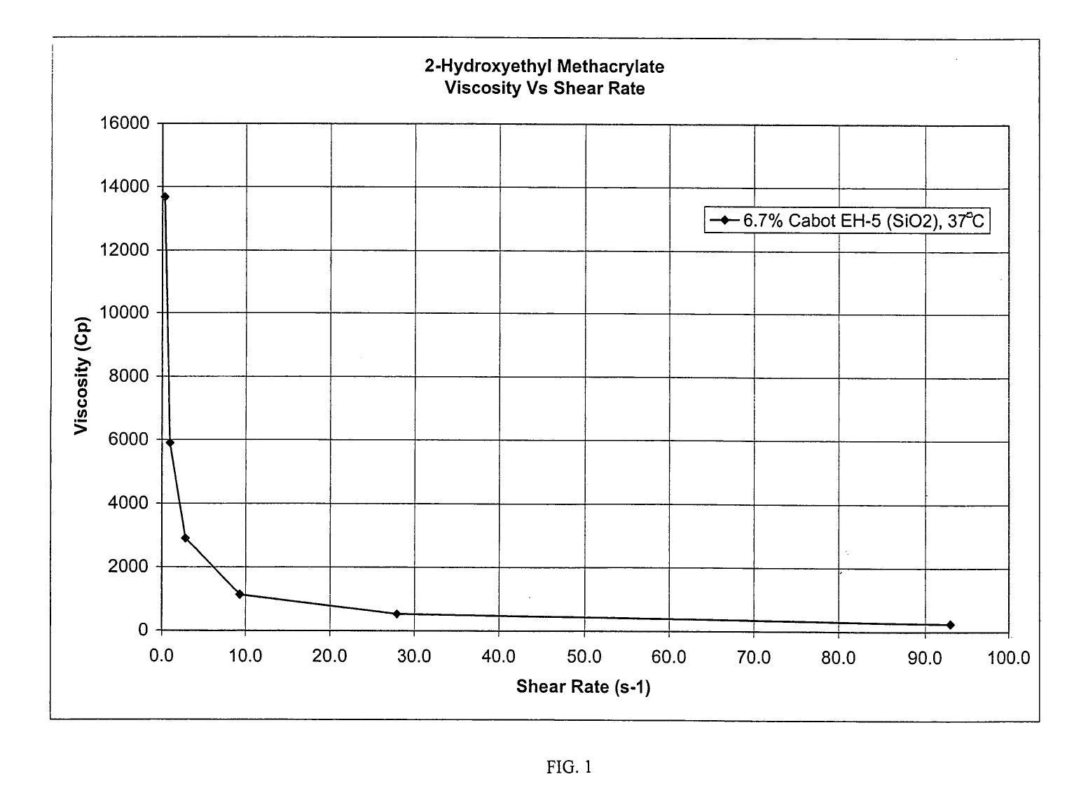

FIG. 1 illustrates the non-Newtonian flow of the composition above. The composition exhibits viscosities under high shear rates that are significantly less than those under low shear rates. It is this characteristic that provides for facile delivery of the composition while maintaining its property of site specific delivery in vivo.

### Example 2

This example illustrates an in vitro application of a rheologically modified embolic composition. This composition is prepared in the manner of Example 1 above and is delivered via a dual lumen catheter into a Y junction modified to have an artificial aneurysm at the juncture. One lumen of the catheter contains the rheologically modified composition and the other lumen contains a water soluble azo initiator, such as Wako VA-044 (Wako Chemicals, Richmond, Va.) for initiating 2-hydroxyethylmethacrylate. While a flow of saline is maintained through the Y junction, the distal tip of a catheter is introduced into the artificial aneurysm and the composition and the initiator was deposited over a sufficient time to fill the anuerysm.

### Example 3

The purpose of this example is to illustrate how an in vivo application of the composition in the treatment of an aneurysm could be accomplished.

A 10-15 kg mongrel dog is anesthetized. Under sterile conditions and with the aid of an operating microscope, an experimental aneurysm is surgically created in the carotid artery using a jugular vein pouch, employing art recognized protocols. After about one week, the aneurysm is embolized with rheologically-modified composition.

Specifically, the femoral arteries are accessed by cut down and introducers and 7 Fr guiding catheters are placed.

For deposition of the rheologically-modified composition, a microcatheter (e.g., Micro Therapeutics, Inc. Rebar 14, with guide wire) is placed through the guiding catheter and is positioned under fluoroscopic guidance so that the catheter tip is in the aneurysmal sac. A microballoon catheter (4-5 mm balloon) is placed in the carotid artery proximal to the aneurysm. Position is confirmed with injection of a liquid contrast agent. The balloon is inflated to slow or arrest blood flow to prevent displacement of the rheologically-modified composition during injection.

Approximately 0.3 to 0.5 cc of a composition, as described in Example 1, is injected into the aneurysm over 1 to 2 minutes to fill the aneurysm space, as well as an appropriate exogenous trigger, such as Wako VA-044. Care is given not to overfill the aneurysm and block the parent artery with polymer. Filling is easily visualized with fluoroscopy due to the presence of contrast agent in the polymer composition. After about 5 minutes, the polymer is fully precipitated and the catheters are removed from the artery.

From the foregoing description, various modifications and changes in the composition and method will occur to those skilled in the art. All such modifications coming within the scope of the appended claims are intended to be included therein.

1. A composition for placement in a mammalian body comprising:
a) a prepolymeric material which thickens and/or solidifies in situ in the presence of an exogenous trigger; and
b) a sufficient amount of a rheological modifier to permit the composition to exhibit thixotropic behavior prior to completion of the thickening and/or solidifying the prepolymeric material.

2. The composition according to claim 1, wherein said prepolymeric material is selected from the group consisting of acrylates, methacrylates, acrylamides, methacrylamides, styrenes, vinyl acetate and acrylonitrile.

3. The composition according to claim 1 wherein said exogenous trigger is selected from the group consisting of light, electromagnetic radiation, ultrasound, mechanical force, magnetic fields, heating or cooling, the introduction of a salt or catalyst, an acid or a base, and another reactive component.

4. The composition according to claim 3, wherein the exogenous trigger is another reactive component.

5. The composition according to claim 4, wherein the prepolymer and the other reactive component comprise a mixture of acrylates, methacrylates, acrylamides, methacrylamides, stryenes, vinyl acetate, acrylonitrile, with one another or with maleic acid, urethane, urethane carbonates, silicone or epoxy.

6. The composition according to claim 1, wherein the rheological modifier is selected from the group consisting of non-particulate rheological modifiers, particulate rheological modifiers and mixtures thereof.

7. The composition according to claim 6, wherein the particulate rheological modifier is selected from the group consisting of silacatious earths, bentonite, organoclays, water-swellable clays, such as lapenite, and silicas such as fumed silica and precipitated, calcium carbonate, titanium dioxide, laminate, titanium oxide, zinc oxide, hydroxyappetite, carbon beads, dispersed fiber, magnetic materials and mixtures thereof.

8. The composition according to claims 6, wherein the non-particulate rheological modifier is selected from the group consisting of polyacrylates, polyalkenes, polyalkyl oxides, polyamides, polycarbonates, cellulosic polymers and copolymers thereof, polydienes, polyesters, polymethacrylates, polysiloxanes, polystyrenes, polyurethanes, polyvinyl ethers, polyvinyl esters, Carbopol, acrylic polymers, cross-linked acrylic polymers, hydroxypropylcellulose, hydroxypropylmethylcellulose, oxidized polyethylene and their copolymers, polyethylene oxide, polyvinylpyrrolidone, associative thickeners, Carrageenan, carboxymethylcellulose, sodium hydroxyethylcellulose, hydroxyethylcellulose, methylcellulose, Guar, Guar derivatives, Locust Bean Gum, Xanthan Gum, and mixtures thereof.

9. The composition according to claim 1, which further comprises a contrast agent.

10. The composition according to claim 9, wherein the contrast agent is water insoluble.

11. The composition according to claim 10, wherein the water insoluble contrast agent is selected from the group consisting of tantalum, tantalum oxide, tungsten, gold, platinum and barium sulfate.

12. The composition according to claim 9, wherein the contrast agent is water soluble.

13. The composition according to claim 12, wherein the water soluble contrast agent is selected from the group consisting of metrizamide, iopamidol, jothalamate socium, jodomide sodium, and meglumine.

14. The composition according to claim 1, wherein said composition further comprises one or more components selected from the group consisting of thickening agents, plasticizers, radioactive agents and surfactants.

15. The composition according to claim 14, wherein said composition further comprises a radioactive agent in a sufficient amount to ablate tissue.

16. The composition according to claim 15, wherein said radioactive agent is selected from the group consisting of ⁹⁰yttrium, ¹⁹²iridium, ¹⁹⁸gold, ¹²⁵iodine, ¹³⁷cesium, ⁶⁰cobalt, ⁵⁵cobalt, ⁵⁶cobalt, ⁵⁷cobalt, ⁵⁷magnesium, ⁵⁵iron, ³²phosphorous, ⁹⁰strontium, ⁸¹rubidium, ²⁰⁶bismuth, ⁶⁷gallium, ⁷⁷bromine, ¹²⁹cesium, ⁷³selenium, ⁷²selenium, ⁷²arsenic, ¹⁰³palladium, ²⁰³lead, ¹¹¹indium, ⁵²iron, ¹⁶⁷thulium, ⁵⁷nickel, ⁶²zinc, ⁶²copper, ²⁰¹thallium and ¹²³iodine.

17. The composition according to claim 14, wherein said composition further comprises a medicament.

18. The composition according to claim 17, wherein said medicament is selected from the group consisting of an angiogenesis inhibiting compound, a steroidal or non-steroidal anti-inflammatory agent, and a thrombotic agent.

19. A method for the site specific delivery of a composition into the body of a mammal which method comprises inserting a delivery device at a targeted site in the mammal and administering via the delivery device a composition according to claim 1 under such conditions that a solid mass is formed in vivo.

20. A method for site specific vascular embolization via a catheter comprising proximal and distal ends wherein the method comprises inserting the distal end of the catheter in the selected vascular site of a mammal, delivering via the catheter a composition of claim 1 to said vascular site under conditions wherein a mass is formed which embolizes the blood vessel.

21. A method for bulking tissue in a mammal which comprises inserting a delivery device into mammalian tissue, delivering via the device a composition according to claim 1 under conditions wherein a solid mass is formed which bulks the tissue.

22. The method according to claim 21 wherein tissue sites suitable for bulking are selected from the group consisting of the suburethral tissue, the periurethreal tissue, soft tissue and sphincters.

23. A method for delivery of a composition comprising a medicament into a mammalian body which method comprises inserting an appropriate delivery device at a targeted site in the patient and then administering via the delivery device a composition according to claim 17 under such conditions that a mass is formed in vivo.

24. A kit of parts comprising:
a) a composition comprising a prepolymeric material which thickens and/or solidifies in situ in the presence of a exogenous trigger, a sufficient amount of a rheological modifier to permit the composition to exhibit thixotropic behavior, and optionally contrast agent; and
b) a delivery device.
